(19) United States
(12) Patent Application Publication
(10) Pub. No.: US 2014/0046240 A1
(43) Pub. Date: Feb. 13, 2014

(54) ELECTRONIC ASSEMBLY FOR IONTOPHORESIS TRANSDERMAL DRUG DELIVERY AND DEVICE THEREOF

(71) Applicant: STMicroelectronics International N.V., Amsterdam (NL)

(72) Inventors: Sneh Anand, New Delhi (IN); Veena Koul, New Delhi (IN); Nitin Agarwal, New Delhi (IN); Amit Gupta, Greater Noida (IN); Alok Kumar Mittal, Greater Noida (IN)

(21) Appl. No.: 13/907,768

(22) Filed: May 31, 2013

(30) Foreign Application Priority Data
Aug. 7, 2012 (IN) 2480/DEL/2012

(51) Int. Cl. A61N 1/30

(52) U.S. Cl. CPC A61N 1/30
USPC 604/20

(57) ABSTRACT

An electronic assembly for iontophoresis transdermal delivery of at least one drug includes an input mechanism configured to receive at least one parameter for iontophoresis transdermal delivery of the at least one drug. The electronic assembly further includes a controller configured to select current intensity and a voltage modulation based on the at least one parameter. The controller is further configured to generate a voltage waveform based on the selected current intensity for a drug delivery period.

### BACKGROUND

1. Technical Field

The present disclosure relates, in general, to iontophoresis transdermal drug delivery devices, and in particular, to an electronic assembly for iontophoresis based transdermal drug delivery and an iontophoresis based transdermal drug delivery device including the electronic assembly.

2. Description of the Related Art

Transdermal drug delivery is used to deliver specific dosages of drugs through the skin and into the bloodstream. However, the success of such drug delivery is limited by the barrier posed by the stratum corneum (SC), which is the outermost layer of the skin. To overcome this barrier, iontophoresis techniques have been developed in the art. Iontophoresis is a non-invasive technique for delivering a drug through the skin using a low intensity current. Typically, the flux of the drug delivered into the skin is in proportion to the current, and hence, different dosages of the drug can be delivered based on the current used.

Iontophoresis based transdermal drug delivery is being exploited for transdermal delivery of drugs. At present, iontophoresis is used for delivery of specific drugs, such as lidocaine and fentanyl, through iontophoresis based transdermal drug delivery devices. Such devices are either pre-programmed or programmed on demand by trained operators for controlled delivery of drugs by adjusting the current used. Iontophoretic devices are generally bulky and involve administering the drug over a long duration, of the order of a few hours. Typically, direct current (DC) is used for providing the current for iontophoresis. However, due to limitations on total amount of current that can be delivered without damaging the skin, the rate and total amount of drug delivered over a drug delivery period is limited.

### BRIEF SUMMARY

This summary is provided to introduce concepts related to a novel electronic assembly for iontophoresis transdermal drug delivery (ITDD) and an ITDD device thereof, and the concepts are further described below in the detailed description. This summary is not intended for use in determining or limiting the scope of the claimed subject matter.

In an embodiment, an electronic assembly is configured to generate a voltage waveform corresponding to a selected current intensity and a selected voltage modulation for iontophoresis transdermal drug delivery (ITDD). The selected current intensity and the selected voltage modulation correspond to at least one parameter of a drug for iontophoresis transdermal delivery received via an input mechanism. In another embodiment, the electronic assembly is included in an ITDD device for iontophoresis transdermal delivery of a drug through the ITDD device. The ITDD device further comprises at least one pair of electrodes configured to receive the voltage waveform from the electronic assembly and apply the voltage waveform for iontophoresis transdermal delivery of the drug. Further, the electronic assembly includes a feedback mechanism, which is configured to regulate the current intensity based on skin impedance during a drug delivery duration.

### BRIEF DESCRIPTION OF THE SEVERAL VIEWS OF THE DRAWINGS

The detailed description is described with reference to the accompanying figures. In the figures, the left-most digit of a reference number identifies the figure in which the reference number first appears. The same numbers are used throughout the drawings to reference like features and components.

FIG. 1 illustrates a network diagram showing the implementation of an iontophoresis transdermal drug delivery device including an electronic assembly, according to one embodiment of the present subject matter;

FIG. 2 shows a block diagram of the electronic assembly for iontophoresis transdermal drug delivery, according to one embodiment of FIG. 1; and

FIG. 3 shows a schematic illustration of a circuit in the electronic assembly for controlled current output, according to one embodiment of FIG. 2.

FIG. 4 shows a method for iontophoresis transdermal delivery of a drug, according to one embodiment of FIG. 2.

### DETAILED DESCRIPTION

Transdermal drug delivery offers many advantages over other conventional routes of drug delivery, such as oral administration, and injections. However, only a few drugs have been successfully developed into formulations suitable for transdermal drug delivery because of the skin barrier. Attempts to facilitate transportation of drug molecules across skin include the use of chemical and physical methods for flux enhancement. Iontophoresis is a technology that enhances drug transport across the skin barrier with the assistance of an electric field. Direct current (DC) iontophoresis with a constant current approach is the most common form of conventional iontophoresis transdermal drug delivery (ITDD).

The DC iontophoresis approach has been successfully applied for administration of small polar molecules. However, the approach results in ion clustering near electrodes in DC-ITDD devices. The ion clustering reduces the rate of drug permeation through the skin, thereby limiting the delivery of high molecular weight compounds, and therapeutic drug molecules which are required to be administered in high doses. The high doses, as known in the art, are doses of approximately ≧500 mg of the appropriate drug. The dosage of drug is also limited due to clinical limitations on total amount of current that can be applied without injuring the skin. Also, burns or irritations at the site of administration of drug are commonly observed due to the application of direct current over a long duration and thus, making the DC-ITDD devices unsuitable for sustained or repeated use. As a result, the usage of DC-ITDD devices has been limited to the administration of specific drugs having small hydrophilic compounds, which have molecular weight less than 3000 Da and some potent bio-therapeutic moieties. Further, such DC-ITDD devices are bulky and are not user friendly as trained operators are required for operating such devices.

The present subject matter discloses an electronic assembly and an ITDD device thereof for administering drugs. The electronic assembly is configured to generate a voltage waveform corresponding to a selected current intensity and a selected voltage modulation, based on a drug to be delivered. In one embodiment, the electronic assembly is used as a research platform for identifying a suitable current intensity, drug delivery period, and a voltage waveform for a specific drug based on a patient profile and a drug administration profile. In another embodiment, the electronic assembly is included in the ITDD device for iontophoresis transdermal delivery of a drug. The ITDD device includes a pair of electrodes for receiving the voltage waveform generated by the electronic assembly and applying across the skin of a patient. The ITDD device further includes a drug delivery reservoir that contains the drug, so as to cause the drug to penetrate the skin of the user. The ITDD device is designed to be portable and does not require a trained operator for its use. Also, the ITDD device is capable of delivering high doses of drug in shorter time as compared to conventional devices.

Through the electronic assembly, the ITDD device can be configured to effectively deliver various drugs using a current intensity, a voltage waveform, and a drug delivery period most suitable for the respective drug. The suitable current intensity, voltage waveform, and drug delivery period are selected based on a patient profile, such as body resistance, age, gender, ethnicity, and so on, and a drug administration profile, such as immediate release of drug, extended or continuous release of drug, timed release of drug, and so on. Thus, the ITDD device enables the usage of iontophoresis technique with a plurality of drugs including drugs having high molecular weight, such as bio-therapeutic molecules. Further, since different waveforms, such as sine wave, square wave, saw tooth wave, and asymmetric waves, and any other desired waveform can be generated by the electronic assembly, the adverse effects associated with use of DC can be substantially eliminated.

The electronic assembly is further configured to maintain the selected current intensity based on skin impedance during the drug delivery period. For this, the electronic assembly includes a feedback mechanism, which facilitates in maintaining a substantially constant current intensity for varying skin impedance without manual intervention. Thus, the ITDD device can provide better control over the amount of drug to be delivered as compared to the conventional devices. This control over the amount of drug to be delivered also helps in reduction in incidences of burns or irritation, thereby making the ITDD device more suitable for sustained or repeated use.

The ITDD device, according to one embodiment of the present subject matter is easy to operate, re-usable, and advantageous especially for paediatric, geriatric, chemotherapy applications, etc., for targeted drug delivery, and for providing instant relief.

The present subject matter is explained in conjunction with the following figures. It should be noted that the description and figures merely illustrate the principles of the present subject matter. It will thus be appreciated that those skilled in the art will be able to devise various arrangements that, although not explicitly described or shown herein, embody the principles of the present subject matter and are included within its spirit and scope. Moreover, all statements herein reciting principles, aspects, and embodiments of the present subject matter, as well as specific examples thereof, are intended to encompass equivalents thereof.

It will be appreciated by those skilled in the art that the words during, while, and when, as used herein, are not exact terms that mean an action takes place instantly upon an initiating action but that there can be some small but reasonable delays such as a propagation delay between the initial action and the reaction, that is initiated by the initial action. Additionally, the word “coupled” is used throughout for clarity of the description and can include either a direct connection or an indirect connection. The descriptions and details of well-known components are omitted for simplicity of the present description.

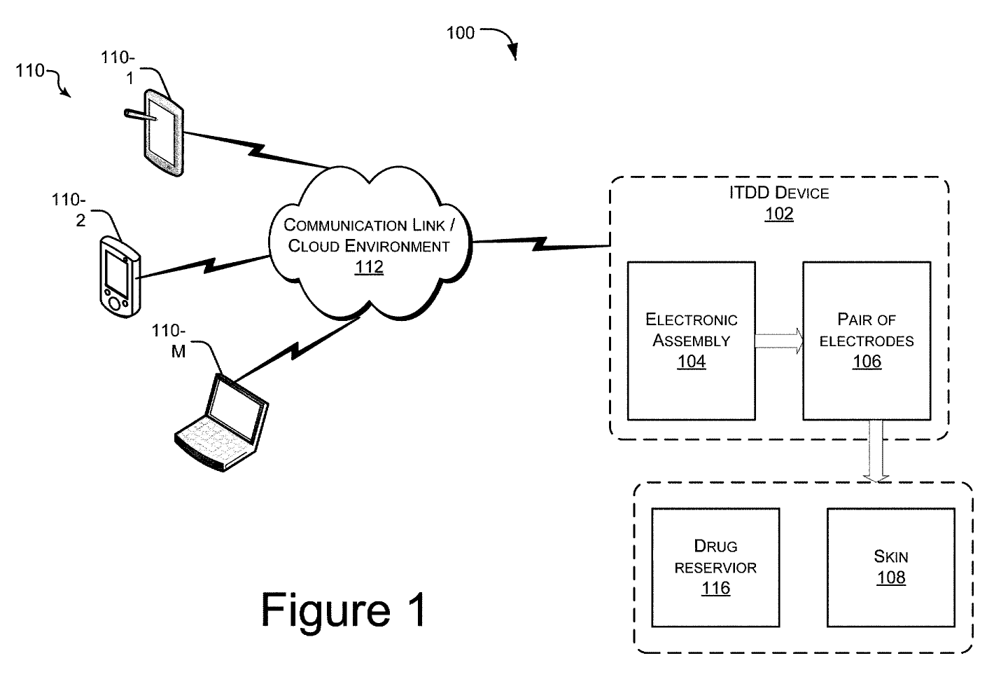

FIG. 1 illustrates a network diagram 100 showing the implementation of an iontophoresis transdermal drug delivery (ITDD) device 102 including an electronic assembly 104 for iontophoresis transdermal delivery of a drug, according to the present subject matter. The electronic assembly 104 is configured to generate a suitable voltage waveform, hereinafter referred to as voltage waveform, having a selected current intensity and a selected voltage modulation, based on a user input, for the iontophoresis transdermal delivery of the drug. In one embodiment, the electronic assembly 104 can be used as a research platform by an industrial user for conducting research and identifying a suitable current intensity, voltage waveform required to effectively deliver a drug into the skin of a patient. The industrial user, according to one embodiment, may include but is not limited to pharmaceutical research division, a research institute, medical device manufacturing company, and the like. The electronic assembly can be used as a stand-alone device or used in conjunction with other suitable devices for conducting research.

In another embodiment, the electronic assembly 104 can be included in the ITDD device 102 for use by a user other than an industrial user, such as a patient, or a medical practitioner. The ITDD device 102 further includes at least one pair of electrodes 106 (hereinafter referred to as pair of electrodes 106) such that at least one of the pair of electrodes 106 can be brought into contact with the site of administration of the drug on a skin 108 of a patient, for example, by strapping the ITDD device 102 to a hand of the patient or placing the ITDD device 102 on the skin 108 using a suitable adhesive or a strap. The ITDD device 102 can be encapsulated in casing such that the ITDD device 102 can be easily, securely and comfortably placed on the required portion of the patient's body. The casing can have a shape similar to or can be flexible enough to conform to a part of the patient's body, such as arms, legs, abdomens, and so on.

In one implementation, a look-up table including a mapping of name of drug, current intensity, voltage waveform, drug delivery period, and dosage for a plurality of drugs is pre-programmed in a memory of the electronic assembly 104. The look up table may be populated based on a patient profile and a drug administration profile for iontophoresis transdermal delivery of the drug. In such a case, the user input can correspond to at least one parameter for iontophoresis transdermal delivery of the drug, such as a name of drug, current intensity, voltage modulation, a dosage of drug, and a drug delivery period. Based on the user input, the electronic assembly 104 can select suitable current intensity and voltage modulation, and generate a voltage waveform for a drug delivery period. Table 1 below indicates examples of various drugs that can be administered by the ITDD device 102. As will be appreciated by those skilled in the art, the implementation of the present subject matter is not limited to the examples of the drugs mentioned herein and other suitable drugs can also be administered by the ITDD device 102.

TABLE 1

| Drug Name | Use |
|---|---|
| Diclofinac | Pain killer |
| Methotrexate | Rheumatoid arthritis, cancer, |
| | paediatric Rheumetisis |
| Rivastagmine | Alzheimer's disease |
| Biotheraputics | Vaccine delivery, gene delivery |
| Insulin | Diabetes |
| Tulobuterol | Asthma transdermal patches |
| Lidocaine | Anaesthetic, Paediatrics |
| Fentanyl | Pain killer |
| Chemotherapy Drugs | Cancer |

In one implementation, the ITDD device 102 can receive the user input from one or more communication devices 110-1, 110-2, . . . 110-M, collectively referred to as communication devices 110, via communication link or cloud environment 112. It will be understood that the communication devices 110 can be wired or wireless devices, and accordingly, the communication link or cloud environment 112 can be wired or wireless. Examples of communication devices include mobile computing devices, such as personal computer, personal digital assistant (PDA), tablet computer, smart phone, integrated circuit card, and so on. Examples of wired communication links include cable connections, such as USB connection, RS232 cable connection, and so on. Examples of wireless communication links include near field communication (NFC), radio frequency communication, Bluetooth, Infra Red, ZigBee, and the like, and long distance communication protocols, such as GSM, CDMA, and the like. In one another example of the wireless communication links, the electronic assembly 104 includes an antenna and receiving electronics. The user input can be received through communication devices communicating via the Internet, World Wide Web, or cloud based environment. A local transmitter near the user can receive the input via the communication link 112 and then transmit it to the antenna and receiver in the electronic assembly 104.

User input can also be provided manually through communication devices 110 coupled to the ITDD device 102 using cable connections. In another implementation, the ITDD device 102 can additionally or alternatively include input devices, such as joystick, touch-screen, push button, keypad, bar code reader, and the like, for receiving user inputs manually. The input devices can be embedded in the ITDD device 102 as an integral part of the ITDD device 102 or can be coupled to the ITDD device 102 as an external part of the ITDD device 102.

Further, the electronic assembly 104 is connected to the pair of electrodes 106 and can provide the voltage waveform to the pair of electrodes 106 for iontophoresis transdermal delivery of the drug. The pair of electrodes 106 can be manufactured using corrosion resistive conductive materials, such as silver, graphite carbon, and so on. Further, electrodes such as gold, platinum, and titanium can also be used. It would be appreciated by those skilled in the art that the material chosen for manufacturing the electrodes should be inert and non-corrosive in nature for avoiding changes in pH of the drug, thereby enhancing the stability of the drug and preventing skin irritation.

In operation, a first electrode of the pair of electrodes 106 can be placed on a drug reservoir 116 containing the drug to be administered, which is in contact with the skin 108. The drug can be in the form of a medication, such as gel based, hydrogel patch, nanoparticles, cream, lotion, ointment, and so on, having ionic charge and can be incorporated in the drug reservoir 116 using methods known in the art. The drug reservoir 116 can be coupled with the first electrode to form a patch for easy application. The first electrode can be directly placed on the skin over a topical application of the drug on the skin without the need of the drug reservoir 116. A second electrode of the pair of electrodes 106 is placed on the skin 108 in the vicinity of the first electrode, thereby forming a closed circuit across the skin 108 and the drug reservoir 116. The circuit can be activated by the patient using a push button or any other input interface on the ITDD device 102 or using the communication device 110 through the communication link 112 so that the voltage waveform is generated by the electronic assembly 104 and applied to the skin 108, and the drug reservoir 116 through the pair of electrodes 106. As a result of the application of the voltage waveform, the drug is delivered into the skin 108 effectively. A plurality of first electrodes of the pair of electrodes 106 can be coupled with a plurality of drug reservoirs which can be placed on the skin at multiple locations to form an array of drug delivery points for more effective release of drug into the skin. The array of drug delivery points can also be formed by coupling a plurality of pair of electrodes with the plurality of drug reservoirs. In one implementation, the circuit can be powered by a battery in the ITDD device 102. The battery can be re-chargeable for repeated use. The ITDD device 102 can be powered by any suitable energy source. Examples of such energy source include AC/DC adaptors, solar cells, super cap, storage capacitors, and the like.

In one example implementation of the ITDD device 102, the first electrode of the pair of electrodes 106 is placed over the drug reservoir 116, and the second electrode of the pair of electrodes 106 is placed over the skin 108 in the vicinity of the drug reservoir 116. The electronic assembly 104 receives the user input regarding at least one parameter for iontophoresis transdermal delivery of the drug through a communication device 110 via the communication link or cloud environment 112. Based on the user input, the electronic assembly 104 generates the voltage waveform having a selected current intensity for a drug delivery period for effectively releasing the drug into the skin 108.

Thus, the ITDD device 102 enables the generation of current in the form of a waveform that is most suitable for administering the specific drug transdermally into the body. Further, the generation of specific waveform for specific drugs through the single ITDD device 102 enables the administration of drugs having smaller molecular weight and also drugs having high molecular weight. Further, the ITDD device 102 can be easily used for administering drug to children and old people due to the miniaturized size of the device and ease of use as compared to oral administration or injecting the drug.

In one implementation, the ITDD device 102 is re-usable. The electronic assembly 104 can be programmed for immediate or extended release of a plurality of drugs to be administered based on patient profile and drug administration profile for a drug. The pair of electrodes 106 can be replaced as required due to standard wear and tear. Thus, the ITDD device 102 is suitable for use in clinics and hospitals also apart from use by individuals.

In one another implementation, the ITDD device 102 can be pre-programmed for only a specific drug such that the ITDD device is used for single use. In yet another implementation, the ITDD device 102 can be programmed for only a specific drug as and when required, such that the ITDD device is used for single use or repeated use.

In yet another implementation, the ITDD device 102 can provide for extended controlled release of drugs as a depot is formed beneath the skin on the administration of the drug. The drug can slowly be released into the blood stream from the depot over an extended period of time. This also helps in achieving a reduction in drug toxicity as the administered drug reaches the site of location in the blood stream immediately and avoids hepatic bypass in comparison to drug toxicity induced via oral administration of drugs.

The ITDD device 102 is user-friendly as the patient can administer the drug on his own without the need of a trained operator. Further, the ITDD device 102 can provide for administering more than one drug thus making the ITDD device 102 suitable for sustained or repeated use. While the ITDD device 102 has been described with reference to usage with a plurality of drugs, it will be understood that the ITDD device 102 can also be configured to administer one type of drug at different dosage levels as will be understood to a person skilled in the art.

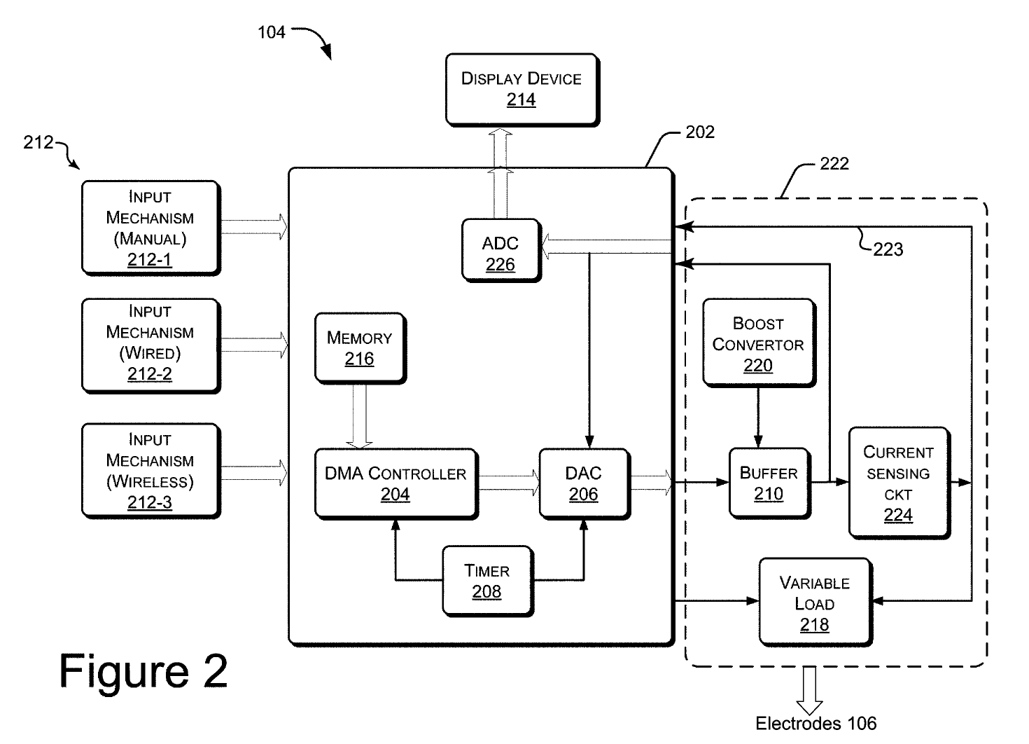

FIG. 2 illustrates a block diagram of the electronic assembly 104 of FIG. 1, according to one embodiment of the present subject matter. As described above, the electronic assembly 104 generates required voltage waveforms for iontophoresis transdermal delivery of drug. The electronic assembly 104 includes a controller 202 for controlling the iontophoresis transdermal drug delivery. Controllers such as a microcontroller, microprocessor, or electronic controller can be used. The controller 202 further includes a direct memory access (DMA) controller 204, a digital to analog convertor (DAC) 206. In one implementation, the controller 202 can also include a timer 208. The DMA controller 204, the DAC 206 and the timer 208 can be either coupled to a controller or embedded into the controller. The DAC 206 is connected to buffer 210, which is then connected to the pair of electrodes 106.

As described above, the electronic assembly 104 can receive a user input corresponding to at least one parameter for iontophoresis transdermal delivery of drug via communication devices 110 and input devices. The user input is received by the electronic assembly 104 through an input mechanism 212. The input mechanism 212 can be, for example, a manual input interface 212-1 or a wireless communication interface 212-3 or a wired communication interface 212-2. In one implementation, the input mechanism 212 can be provided through the input devices, such as joystick, push key, bar code reader and so on, coupled to the electronic assembly 104 via the manual input interface. In another implementation, the input mechanism 212 can be provided through a wireless communication interface, such as NFC, Bluetooth, IR, radio frequency communication, and the like, or via long distance communication protocols such as GSM, CDMA, and the like. In such implementation, a mobile computing device, such as smart phone, tablet, PC can be communicatively coupled to the electronic assembly 104 via wireless communication interface. In one example of such implementation, a mobile computing device can be communicatively coupled to the electronic assembly 104 via wireless communication interface over a cloud based environment. In another implementation, the input mechanism 212 can be provided through a wired communication interface, such as USB port, RS232 port, and so on. In such implementation, the mobile computing device can be communicatively coupled to the electronic assembly 104 using cable connections such as USB cable, RS232 cable, and so on.

Further, a display device 214, such as TFT, LCD, LED displays, OLED display, E-Paper, and the like, can also be coupled to the electronic assembly 104 for displaying various parameters for monitoring purposes. In one implementation, an audio indicator, such as a buzzer, or an alarm can be coupled with the electronic assembly 104 for indicating various stages during the iontophoresis transdermal delivery of the drug. In one another implementation, a visual indicator such as LED can be coupled with the electronic assembly 104 for indicating various stages during the iontophoresis transdermal delivery of the drug.

In one implementation, a user can provide at least one parameter for iontophoresis transdermal delivery of a drug as a user input to the electronic assembly 104 using the input mechanism 212. The parameters for iontophoresis transdermal delivery of the drug may include name of the drug, dosage of the drug, required current intensity, required voltage modulation, and so on. In one example of such implementation, a mobile application can be downloaded onto a mobile computing device of the user and the user input can be provided through the mobile application via a wireless interface, such as Bluetooth directly to the electronic assembly 104. In another example of an implementation, the user can provide the user input manually through input devices 212, such as keypad, joy stick, touch screens, and the like, coupled to the electronic assembly 104 via manual input interface. In another example of such implementation, the user can provide the user input through a computing device coupled to the electronic assembly 104 via wired interfaces, such as USB port, RS232 port, and the like.

Based on the at least one parameter received as the user input, the DMA controller 204 can access a look-up table stored in a memory 216. The memory 216 can be embedded in the controller 202 or can be externally coupled to the controller 202. The look-up table can include a mapping of name of the drug, dosage of the drug, required current intensity to be generated, required voltage modulation to be used, and a drug delivery period for which the voltage waveform is to be generated for each of a plurality of drugs that can be administrated using the iontophoresis approach. Thus, the controller 202 selects current intensity, voltage modulation and drug delivery period, also referred to collectively as selected data from the look-up table based on the at least one parameter received as the user input. The controller 202 generates a required voltage waveform based on the selected data for administering the drug. The controller 202 can also set a timer 208 for the drug delivery period for generating the required voltage waveform. The selected voltage waveform can be generated by the controller 202 using methods as known in the art.

The present subject matter is not limited to generation of the required voltage waveform by a controller in the electronic assembly as described herein. The generation of required voltage waveform for iontophoresis based transdermal drug delivery in the electronic assembly can be achieved by any suitable digital circuit, analog circuit, or a combination of digital and analog circuit as known in the art.

The electronic assembly 104 as described above can be manufactured either as System on Chip (SoC) or Application Specific Integrated Circuit (ASIC) such that the desired functionality can provided through a single customized integrated circuit (IC) instead of an assembly of electronic components on a circuit board. Thus, the electronic assembly 104 can be one of a SoC, an ASIC, and an assembly of electronic components on a circuit board.

In one embodiment, the electronic assembly 104 can be used as a research platform by an industrial user, such as a pharmaceutical research division, medical device manufacturing company, research institute, and the like. Using the electronic assembly 104, the industrial user can conduct research and identify suitable current intensity, and voltage waveform for effective iontophoresis transdermal delivery of a drug. The industrial user can provide at least one parameter for iontophoresis transdermal delivery of a drug such as current intensity, voltage modulation, drug delivery period, for a drug as a user input to the electronic assembly 104. Based on the user input, a required current intensity and a required voltage modulation can be selected. Based on the selected current intensity, the electronic assembly 104 generates a required voltage waveform. During the research various parameters, such as clinical limitation on current intensity, penetration of the drug, and so on are monitored. Further, the current intensity, the voltage modulation, the drug delivery period can be modulated and the voltage waveform can be changed based on the monitored parameters. Thus, the industrial user is able to identify the suitable current intensity, drug delivery period, and the voltage waveform for a specific drug based on a patient profile and drug administration profile by using the electronic assembly 104 as a research platform. Further, the industrial user can program an ITDD device 120 containing an electronic assembly according to specific drugs for use by patients.

In another embodiment, the electronic assembly 104 can be included in the ITDD device 102. A user such as a patient or a medical practitioner can provide at least one parameter for iontophoresis transdermal delivery of a drug as a user input to the electronic assembly 104 using the input mechanism 212. The parameters for iontophoresis transdermal delivery of the drug may include name of the drug, dosage of the drug, required current intensity, required voltage modulation, and so on. Based on the user input, the controller 202 access at least one lookup table and selects a required current intensity and voltage modulation. The controller 202 then generates a required voltage waveform as described earlier. The DAC 206 then generates an analog signal corresponding to the required voltage waveform as an output and provides the signal to the pair of electrodes 106 via a buffer circuit 210 for applying the required voltage waveform as described earlier. The DAC is therefore a voltage generation circuit outputting an analog voltage corresponding to the waveform generated by the controller.

In one implementation, the electronic assembly 104 in the ITDD device 102 can be pre-programmed for a specific drug and a specific dosage pattern, and can also be re-programmed by a user such as a medical practitioner via the input mechanism 212 so that the ITDD device 102 is used for delivering a plurality of drugs. In an example, the medical practitioner can decide on a drug profile for a drug based on a patient profile. The drug profile may include various parameters, such as current intensity, voltage waveform, required drug delivery period, name of drug, dosage of drug, and so on. A medical practitioner can also download the drug profile from a website. The medical practitioner can upload the drug administration profile into the memory of the electronic assembly 104 via mobile application downloaded into a mobile computing device of the medical practitioner. In yet another implementation, the ITDD device 102 can be programmed based on specific requirements of industrial users such as a pharmaceutical or a drug company, medical electronics company, any end-product designing company considering various business and regional demands. For this, the ITDD devices 102 can be coupled with communication devices or input devices using the methods as described above. Thus, using the methods as described herein any ITDD device 102 can be tailored/scaled for delivering a single drug or a plurality of drugs based on application parameters, such as drug, dosage, sex, age, ethnicity, and other parameters necessary for drug delivery.

The controller 202 is further coupled to a sensor to measure a variable load 218 across the pair of electrodes 106 at the site of drug administration. The variable load 218 corresponds to the change in impedance of the skin 108 over the duration of application of the required voltage waveform. As described earlier, the controller 202 is coupled to the buffer 210 such that the output of the DAC 206 is coupled to an input of the buffer 210 to prevent the DAC 206 from getting damaged due to the variable load 218. The buffer 210 is further coupled to a boost convertor 220. The boost convertor 220 helps to increase the output voltage signal from buffer 210.

In one implementation, a feedback mechanism 222 comprising a current sensing circuit 224 and a voltage amplifier (not shown in the figure) is coupled with the DAC 206 to regulate the current intensity so that a substantially constant current intensity is provided across the variable load 218 without any manual intervention. In one example, the variable load 218 is measured continually during the application of the required voltage waveform and a feedback input is fed to the controller 202, via bus 223, which adjusts the output voltage amplitude based on the feedback input. The bus 223 can be a two-way bus or a one-way bus. In another implementation, the variable load 218 is measured continually during the application of the voltage waveform and the output voltage amplitude is adjusted manually, for example using a potentiometer, based on the measured variable load 218. The current flowing through variable load 218 is also given as an input to an analog-to-digital convertor (ADC) 226 through the current sensing circuit 224 via bus 223 and the voltage amplifier for displaying a value of actual current flowing through the ITDD device 102 on the display device 214. The output of the buffer 210 can also be input to the ADC 226. The display device 214 can also display the modulation used, and the frequency and amplitude of the required voltage waveform generated.

Thus, a low cost effective ITDD device 102 can be obtained that consumes less power for operation. In one implementation, the electronic assembly 104 and in turn the ITDD device 102 is powered by a battery, which can last for a long time period of the order of a few hours. Further, rechargeable batteries can be used for powering the ITDD device 102. The batteries used for powering the ITDD device 102 can be thin film batteries, re-chargeable batteries, and the like, as known in the art.

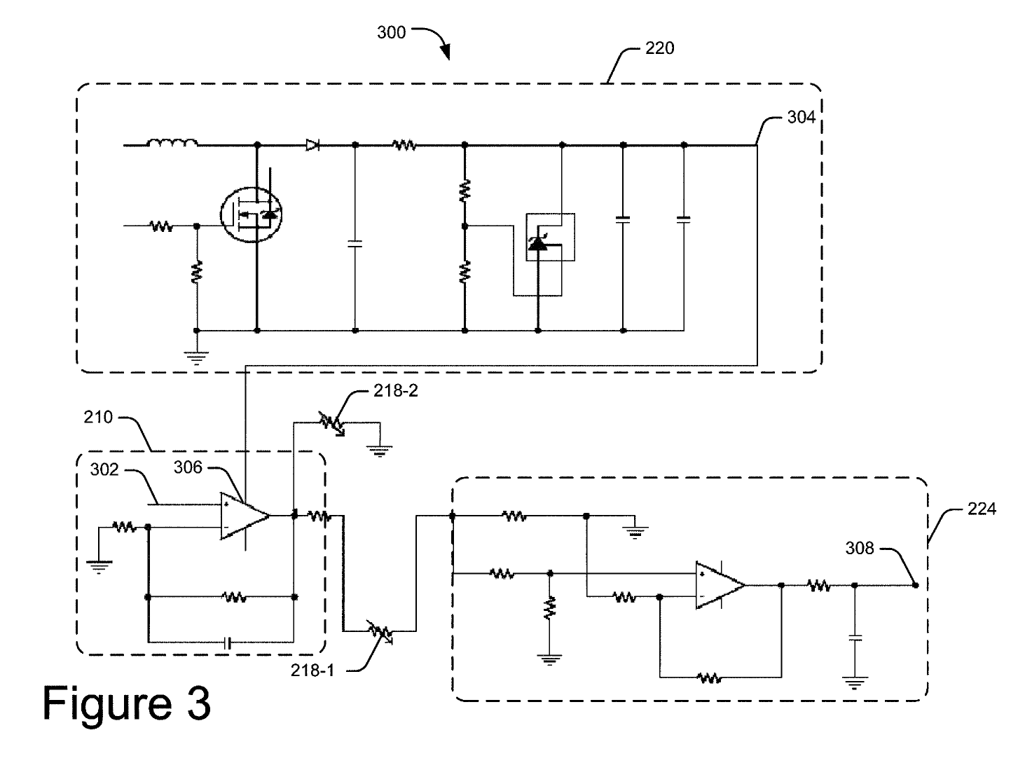

FIG. 3 provides a schematic illustration of a circuit 300 comprising the boost convertor 220, the buffer 210 and the current sensing circuit 224 coupled to the controller 202 of FIG. 2. The circuit 300 provides better control over the amount of drug to be delivered. As described in FIG. 2, the controller 202 generates the required voltage waveform having the selected current intensity and the selected voltage modulation based on the user input. The DAC 206 provides as an analog signal corresponding to the required voltage waveform to the buffer 210 at 302. Further, the boost convertor 220 provides a boosted output at 304 and provides the boosted output as an additional input to the buffer 210 at 306. The combined output from the buffer 210 is then fed to the pair of electrodes 106.

In one implementation, the current sensing circuit 224 measures the variable load 218-1 and provides the feedback input to ADC 226 at 308 for automatic control of the voltage applied. In another implementation, the variable load 218-2 is measured and applied voltage is adjusted based on the measured variable load 218-2 for manual control of the voltage applied. The controller 202 then generates the required amount of voltage amplitude at output of DAC 206.

The present subject matter is not limited to the topology of circuit 300 as described herein.

In one embodiment, the circuit 300 is an external circuit that permits custom selection of the variable components. In other embodiments, one or more of the components of circuit 300 are part of the controller 202, such as the amplifiers, Zener diode, and some of the resistors, while other components are external to the controller 202. The circuit 300 can include other topologies suitable for providing better control over the amount of drug to be delivered and can be designed as per design requirements.

The circuit shown in FIG. 3 has a combination of resistors and capacitors to appropriately bias the components and provide feedback to the amplifiers. While no specific values are given, those of skill in the art would be able to select the appropriate values for proper operation of the circuit to achieve the purposes as taught herein. Furthermore it is not required to use the specific combination of inductors, capacitors, and resistors for the circuit, and in particular, the circuit of FIG. 3 is given as one acceptable example to accomplish the feedback as called for herein, and many other different specific circuits could achieve this feedback to the controller 202. For example, in one embodiment, the variable load sensor is not required, while in other embodiments, different techniques to sense the current or the impedance may be used.

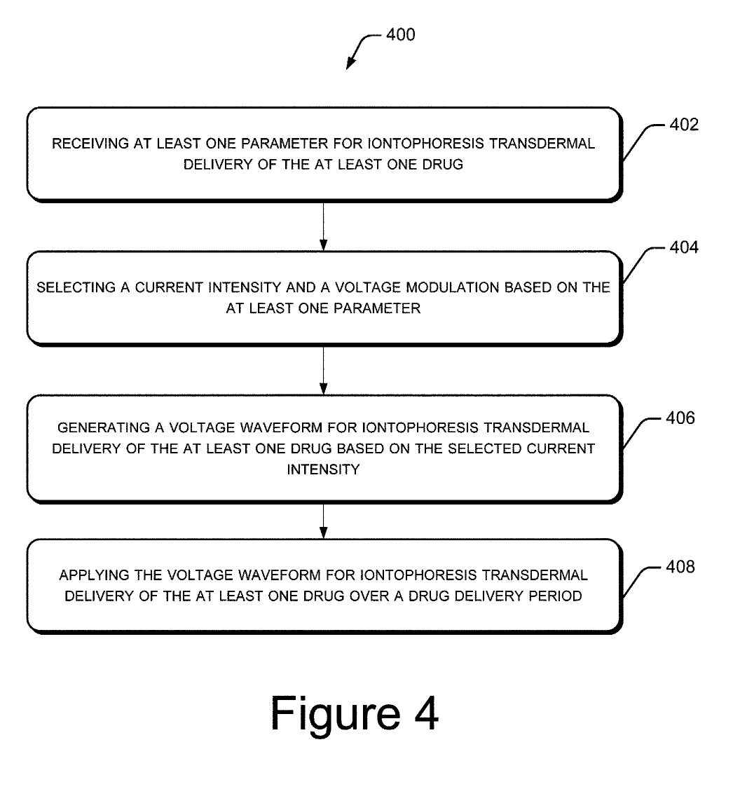

Referring to FIG. 4, a method 400 for iontophoresis transdermal delivery of drug is illustrated. The order in which the method 400 is described is not intended to be construed as a limitation, and any number of the described method blocks can be combined in any order to implement the methods, or alternative methods. Additionally, individual blocks may be deleted from the method 400 without departing from the spirit and scope of the subject matter described herein. Furthermore, the methods can be implemented in any suitable hardware, software, firmware, or combination thereof.

At block 402, at least one parameter of a drug for iontophoresis transdermal delivery is received by the input mechanism 202 of the electronic assembly 104. The at least one parameter of the drug is selected from a name of the drug, current intensity, voltage modulation, dosage of the drug, and drug delivery period of the drug. In one implementation, the at least one parameter is received via a communication link such as near field communication, radio frequency communication, Bluetooth, Infra Red, ZigBee, GSM, CDMA, USB, and RS232.

At block 404, a current intensity and voltage modulation for iontophoresis transdermal delivery of the drug is selected based on at least one parameter. In one implementation, a table including a mapping of a name of a drug, current intensity, voltage modulation is accessed for selection of the current intensity and the voltage modulation.

At block 406, a selected voltage waveform is generated for iontophoresis transdermal delivery of the at least one drug based on the selected current intensity and voltage modulation.

At block 408, the generated voltage waveform is applied for iontophoresis transdermal delivery of the at least one drug over a drug delivery period. In one implementation, the selected current intensity is maintained at a substantially constant value based on variations in skin impedance during the drug delivery period.

In particular, the variable load sensor 218 and/or the current sensor 224 will obtain data on the current amplitude, the voltage at the electrodes, and the impedance of the skin. If the impedance of the skin changes, the feedback bus 223 will carry data to the controller 202 so that the voltage waveform and/or applied current can be changed. If the patient wearing the ITDD begins to sweat, as may happen if exercising or due to local heat, the skin impedance will go down, and the current will increase if the voltage remains the same.

The current sensing circuit will sense this change and provide feedback to the controller 202 to either modify the voltage waveform, limit the current, or both. This is just one example of a benefit of the present embodiment, and others can be achieved with this design.

Although embodiments of the iontophoresis based transdermal drug delivery device have been described in language specific to structural features and/or methods, it is to be understood that the subject matter described herein is not necessarily limited to the specific features or methods described. Rather, the specific features and methods are disclosed as exemplary implementations for the iontophoresis based transdermal drug delivery device.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An electronic assembly for iontophoresis transdermal delivery of at least one drug, the electronic assembly comprising:
an input configured to receive at least one parameter for the iontophoresis transdermal delivery of the at least one drug; and
a controller configured to:
select at least one of a current intensity and a voltage modulation based on the at least one parameter; and
generate the voltage waveform, based on the selected current intensity, for the iontophoresis transdermal delivery of the at least one drug for a drug delivery period.

2. The electronic assembly of claim 1, further comprising a feedback circuit configured to maintain the selected current intensity at a substantially constant value based on variations in skin impedance during the drug delivery period.

3. The electronic assembly of claim 1, wherein the at least one parameter is selected from one of a name of the at least one drug, a dosage of the at least one drug, the drug delivery period, the current intensity, and the voltage modulation.

4. The electronic assembly of claim 3, wherein the at least one parameter is selected based on a patient profile and a drug administration profile.

5. The electronic assembly of claim 1, wherein the input includes an interface from at least one of a near field communication, radio frequency communication, Bluetooth, Infra Red, ZigBee, GSM, CDMA, USB, and RS232, for receiving the at least one parameter.

6. The electronic assembly of claim 1, implemented as one of a System on Chip, an Application Specific Integrated Circuit, and an assembly of electronic components in a circuit board.

7. A iontophoresis transdermal drug delivery device configured for iontophoresis transdermal delivery of at least one drug to a patient, the iontophoresis transdermal drug delivery device comprising:
an electronic assembly configured to:
receive at least one parameter for the iontophoresis transdermal delivery of the at least one drug;
select a current intensity and a voltage modulation based on the at least one parameter; and
generate a voltage waveform, based on the selected current intensity, for the iontophoresis transdermal delivery of the at least one drug for a drug delivery period; and
at least one pair of electrodes configured to receive the generated voltage waveform from the electronic assembly and to apply the voltage waveform for the iontophoresis transdermal delivery of the at least one drug for the drug delivery period.

8. The iontophoresis transdermal drug delivery device of claim 7, wherein the electronic assembly is further configured to maintain the selected current intensity at a substantially constant value based on variations in skin impedance during the drug delivery period.

9. The iontophoresis transdermal drug delivery device of claim 8, wherein the electronic assembly comprises an input for receiving the at least one parameter.

10. The iontophoresis transdermal drug delivery device of claim 9, wherein the input includes an interface including at least one of a near field communication, radio frequency communication, Bluetooth, Infra Red, ZigBee, GSM, CDMA, USB, and RS232.

11. The iontophoresis transdermal drug delivery device of claim 7, wherein the electronic assembly comprises at least one lookup table for selecting a current intensity, and a voltage waveform, wherein the at least one lookup table includes a mapping of name of a drug, dosage, a current intensity, a voltage modulation, a voltage waveform, and a predetermined drug delivery period for iontophoresis transdermal delivery of the at least one drug.

12. The iontophoresis transdermal drug delivery device of claim 7, wherein the iontophoresis transdermal drug delivery device is reconfigured for the iontophoresis transdermal delivery of a second drug based on a plurality of parameters for the iontophoresis transdermal delivery of the second drug received by the input mechanism.

13. A method for iontophoresis transdermal delivery of at least one drug to a patient, the method comprising:
receiving at least one input parameter for the iontophoresis transdermal delivery of the at least one drug;
selecting a current intensity and a voltage modulation based on the at least one input parameter;
generating a voltage waveform for the iontophoresis transdermal delivery of the at least one drug based on the selected current intensity and
applying the voltage waveform for the iontophoresis transdermal delivery of the at least one drug over a drug delivery period.

14. The method of claim 13, further comprising maintaining the selected current intensity at a substantially constant value based on variations in skin impedance during the drug delivery period.

15. The method of claim 13, wherein the at least one parameter is received via a communication link from one of a near field communication, radio frequency communication, Bluetooth, Infra Red, ZigBee, GSM, CDMA, USB, and RS232.

16. A device comprising:
an input configured to receive an iontophoresis input parameter from a user;
a controller coupled to the input and configured to receive the input parameter from the input and to select a current intensity based on the input parameter; and
a voltage generation circuit coupled to the controller, configured to generate a voltage waveform based on the selected current intensity and to implement iontophoretic transdermal delivery of a drug by applying the voltage waveform to at least one electrode.

17. The device of claim 16 wherein the voltage generation circuit includes a digital to analog converter.

18. The device of claim 16 comprising a memory coupled to the controller.

19. The device of claim 18 wherein the controller is configured to access a database stored in the memory to select the voltage waveform based on the input parameter.

20. The device of claim 16 comprising a feedback circuit coupled to the controller, the feedback circuit configured to provide to the controller an indication of a current passed through the at least one electrode.
